(19) United States
(12) Patent Application Publication
(10) Pub. No.: US 2019/0151307 A1
(43) Pub. Date: May 23, 2019

(54) METHODS OF TREATING PATIENTS CO-INFECTED WITH A VIRUS AND TUBERCULOSIS

(71) Applicant: Gilead Sciences, Inc., Foster City, CA (US)

(72) Inventor: Joseph Marcello Custodio, San Francisco, CA (US)

(21) Appl. No.: 16/168,472

(22) Filed: Oct. 23, 2018

Related U.S. Application Data
(60) Provisional application No. 62/638,042, filed on Mar. 2, 2018.
(60) Provisional application No. 62/576,554, filed on Oct. 24, 2017.

(51) Int. Cl. A61K 31/496; A61P 31/06; A61P 31/18; A61K 31/506; A61K 9/20; A61K 31/675

(52) U.S. Cl. CPC A61K 31/496; A61P 31/06; A61K 31/675; A61K 31/506; A61K 9/20; A61P 31/18

(57) ABSTRACT

The disclosure describes methods for the treatment of patients co-infected with a virus and tuberculosis (TB), wherein the patient receives a therapeutically effective amount of tenofovir alafenamide (TAF) and a therapeutically effective amount of an antimycobacterial agent such as rifampin (RIF).

### FIELD

The disclosure describes methods for the treatment of patients co-infected with a virus and tuberculosis (TB), wherein the patient receives a therapeutically effective amount of tenofovir alafenamide (TAF) and a therapeutically effective amount of an antimycobacterial agent such as rifampin (RIF).

### BACKGROUND

Tuberculosis is the leading cause of death globally in HIV-infected patients. HIV-1 infected patients are more likely to develop active tuberculosis (TB) compared to those who do not have HIV-1 infection. Generally, co-infected patients are treated for both infections at the same time.

Tenofovir alafenamide (GS-7340, TAF) is a nucleotide reverse transcriptase inhibitor and a prodrug of tenofovir (TFV). TAF is metabolized intracellularly to the active metabolite, tenofovir diphosphate (TFV-DP), a competitive inhibitor of HIV-1 reverse transcriptase (RT) that terminates the elongation of the viral DNA chain. The intracellular metabolism of TAF and TFV are consistent with the 600-fold enhancement in anti-HIV activity in cell culture of TAF over TFV. TAF is FDA-approved as Vemlidy® for the treatment of chronic hepatitis B invention (HBV). TAF is also a component in Genvoya®, Descovy®, Odefsey®, Symtuza®, each indicated in the treatment of HIV-1 infection. TAF is also a component of a fixed dose combination therapy (including bictegravir (BIC) and emtricitabine (FTC)) for the treatment of HIV-1 infection, which is currently under review at the FDA.

Rifampin is an antimycobacterial that inhibits bacterial DNA-dependent RNA synthesis, and is indicated in the treatment of all forms of tuberculosis as part of a multicomponent antibacterial regimen. However, RIF is a potent inducer of drug metabolizing enzymes including CYP3A4 and UGT1A1, and of efflux transporters such as P-glycoprotein (P-gp). Thus, coadministration of RIF with drugs that are substrates of human transporters like P-gp is expected to adversely affect the effectiveness of the drug.

TAF is a substrate of the human drug transporters P-gp and breast cancer resistance protein (BCRP). Prescribing information for Vemlidy® indicates that coadministration of TAF and RIF is not recommended.

Thus, there is an important need for antiviral agents in combination with antimycobacterial agents like RIF that can be coadministered to patients without significantly compromising therapeutic efficacy of either active agent.

### SUMMARY

The present disclosure provides a method of treating a subject co-infected with a virus and tuberculosis (TB), comprising administering a therapeutically effective amount of tenofovir alafenamide (TAF) and a therapeutically effective amount of an antimycobacterial agent to the subject. In some embodiments, the TAF is administered twice daily. In some embodiments, the TAF is administered once daily. In some embodiments, the virus is selected from HIV and HBV. In some embodiments, the virus is HIV. In some embodiments, the virus is HBV.

In some embodiments, the antimycobacterial agent is selected from the group consisting of rifampin (rifampicin; RIF), rifabutin, rifapentine, isoniazid, ethambutol, pyrazinamide, dapsone, streptomycin, p-amino-salicylate, ethionamide, cycloserine, closerin, capreomycin, viomycin, enviomycin, amikacin, kanamycin, ciprofloxacin, levofloxacin, moxifloxacin, clofazamine, ethionamide, prothionamide, clarithromycin, linezolid, thioacetazone, thioridazine, R207910, and terizidone. In some embodiments, the antimycobacterial agent is RIF. In some embodiments, the antimycobacterial agent is administered at a 600 mg daily dose. In some embodiments, the antimycobacterial agent is administered once daily. In some embodiments, the daily dose of antimycobacterial agent is administered together with the first daily dose of TAF. In some embodiments, the TAF is administered at a 25 mg dose twice daily.

In some embodiments, the method further comprises administering one or more additional therapeutic agents selected from bictegravir, emtricitabine, elvitegravir, cobicistat, atazanavir, ritonavir, lopinavir, darunavir, rilpivirine, efavirenz, saquinavir, fosamprenavir and tipranavir. In some embodiments, the one or more additional therapeutic agents are bictegravir and emtricitabine. In some embodiments, the additional therapeutic agent is emtricitabine. In some embodiments, at least one of the daily doses of TAF is administered together with the one or more additional therapeutic agents. In some embodiments, a single tablet comprising TAF, bictegravir, and emtricitabine is administered to the subject twice daily. In some embodiments, the single tablet comprises 25 mg TAF, 50 mg bictegravir, and 200 mg emtricitabine. In some embodiments, a single tablet comprising TAF and emtricitabine is administered to the subject twice daily. In some embodiments, the single tablet comprises 25 mg TAF and 200 mg emtricitabine. In other embodiments, the single tablet comprises 10 mg TAF and 200 mg emtricitabine. In some embodiments, a single tablet comprising TAF and emtricitabine is administered to the subject twice daily and a second tablet comprising bictegravir or dolutegravir is administered to the subject once daily. In some embodiments, a single tablet comprising TAF and emtricitabine is administered to the subject twice daily and a second tablet comprising bictegravir is administered to the subject once daily. In some embodiments, a single tablet comprising TAF and emtricitabine is administered to the subject twice daily and a second tablet comprising dolutegravir is administered to the subject once daily.

One embodiment provides a method of treating a subject co-infected with HBV or HIV and tuberculosis (TB), comprising administering to the subject: a single tablet twice daily comprising 25 mg TAF, 50 mg bictegravir, and 200 mg emtricitabine; and a once daily dose of 600 mg RIF. In some embodiments, the viral infection is HIV.

One embodiment provides a method of treating a subject co-infected with HBV or HIV and tuberculosis (TB), comprising administering to the subject: a single tablet twice daily comprising 25 mg TAF and 200 mg emtricitabine; and a once daily dose of 600 mg RIF. In some embodiments, the viral infection is HIV.

One embodiment provides a method of treating a subject co-infected with HBV or HIV and tuberculosis (TB), comprising administering to the subject: a single tablet twice daily comprising 10 mg TAF and 200 mg emtricitabine; and a once daily dose of 600 mg RIF. In some embodiments, the viral infection is HIV.

One embodiment provides a method of treating a subject co-infected with HBV or HIV and tuberculosis (TB), comprising administering to the subject: a single tablet twice daily comprising 25 mg or 10 mg TAF, and 200 mg emtricitabine; a once daily dose of 50 mg bictegravir or 50 mg dolutegravir; and a once daily dose of 600 mg RIF. In some embodiments, the viral infection is HIV.

One embodiment provides a method of treating a subject co-infected with HBV or HIV and tuberculosis (TB), comprising administering to the subject: a single tablet twice daily comprising 25 mg TAF, and 200 mg emtricitabine; a once daily dose of 50 mg bictegravir; and a once daily dose of 600 mg RIF. In some embodiments, the viral infection is HIV.

One embodiment provides a method of treating a subject co-infected with HBV or HIV and tuberculosis (TB), comprising administering to the subject: a single tablet twice daily comprising 10 mg TAF, and 200 mg emtricitabine; a once daily dose of 50 mg bictegravir; and a once daily dose of 600 mg RIF. In some embodiments, the viral infection is HIV.

One embodiment provides a method of treating a subject co-infected with HBV or HIV and tuberculosis (TB), comprising administering to the subject: a single tablet twice daily comprising 25 mg TAF, and 200 mg emtricitabine; a once daily dose of 50 mg dolutegravir; and a once daily dose of 600 mg RIF. In some embodiments, the viral infection is HIV.

One embodiment provides a method of treating a subject co-infected with HBV or HIV and tuberculosis (TB), comprising administering to the subject: a single tablet twice daily comprising 10 mg TAF, and 200 mg emtricitabine; a once daily dose of 50 mg dolutegravir; and a once daily dose of 600 mg RIF. In some embodiments, the viral infection is HIV.

In another embodiment of the provided methods, the daily TAF plasma exposure is not reduced by more than about 25% compared with the daily TAF plasma exposure measured for the same treatment but wherein the TAF is administered once daily in the absence of the antimycobacterial agent. In another embodiment, the daily TAF plasma exposure is not reduced by more than about 20% compared with the daily TAF plasma exposure measured for the same treatment but wherein the TAF is administered once daily in the absence of the antimycobacterial agent. In another embodiment, the daily TAF plasma exposure is not reduced by more than about 15% compared with the daily TAF plasma exposure measured for the same treatment but wherein the TAF is administered once daily in the absence of the antimycobacterial agent.

In one embodiment of the provided methods, the daily tenofovir (TFV) plasma exposure is not increased by more than 20% compared with the daily tenofovir (TFV) plasma exposure measured for the same treatment but wherein the TAF is administered once daily in the absence of the antimycobacterial agent. In another embodiment, the daily tenofovir (TFV) plasma exposure is not increased by more than 10% compared with the daily tenofovir (TFV) plasma exposure measured for the same treatment but wherein the TAF is administered once daily in the absence of the antimycobacterial agent. In another embodiment, the daily tenofovir (TFV) plasma exposure is the same as or less than that measured for the same treatment but wherein the TAF is administered once daily in the absence of the antimycobacterial agent.

In one embodiment of the provided methods, the mean steady-state intracellular TFV-DP trough concentration is at least about 85 fmol/10⁶ cells. In another embodiment, the mean steady-state intracellular TFV-DP trough concentration, is at least about 200 fmol/10⁶ cells. In another embodiment, the mean steady-state intracellular TFV-DP trough concentration, is at least about 300 fmol/10⁶ cells. In another embodiment, the mean steady-state intracellular TFV-DP trough concentration, is at least about 350 fmol/10⁶ cells.

In one embodiment of the methods provided herein, the daily TFV-DP intracellular exposure is not reduced by more than about 30% compared with the daily TFV-DP intracellular exposure measured for the same treatment but wherein the TAF is administered once daily in the absence of the antimycobacterial agent. In another embodiment, the daily TFV-DP intracellular exposure is not reduced by more than about 25% compared with the daily TFV-DP intracellular exposure measured for the same treatment but wherein the TAF is administered once daily in the absence of the antimycobacterial agent. In another embodiment, the daily TFV-DP intracellular exposure is not reduced by more than about 20% compared with the daily TFV-DP intracellular exposure measured for the same treatment but wherein the TAF is administered once daily in the absence of the antimycobacterial agent.

### BRIEF DESCRIPTION OF THE DRAWINGS

FIG. 1 is a graph showing mean (SD) TAF concentration (ng/mL) vs. time in study subjects administered either B/F/TAF QD or B/F/TAF BID+RIF.

FIG. 2 shows TFV AUCₜₐᵤ values (ng·h/mL) in study subjects administered B/F/TAF QD, B/F/TAF BID+RIF, or another TDF-containing regimen.

FIG. 3 is a graph showing intracellular concentrations of TFV-DP in study subjects administered either B/F/TAF QD or B/F/TAF BID+RIF.

FIG. 4 is a graph showing mean (SD) BIC plasma concentration (ng/mL) vs. time (0-96 hours) profiles for treatment with either a single dose of BIC or BIC with RIF.

FIG. 5 is a graph showing mean TAF plasma concentration (ng/mL) vs. time (0-24 hours) profiles in healthy subjects after administration of either a single dose of TAF/F or TAF/F with RIF.

FIG. 6 is a graph showing mean TFV plasma concentration (ng/mL) vs. time (0-24 hours) profiles in healthy subjects after administration of either a single dose of TAF/F or TAF/F with RIF.

FIG. 7 shows the mean PBMC concentration of TFV (fmol/million cells) over 24 hours in healthy subjects administered either a single dose of TAF/F or TAF/F with RIF.

FIG. 8 shows the mean PBMC concentration of TFV (fmol/million cells) over 24 hours in healthy subjects administered either a single dose of TDF or TAF/F with RIF.

FIG. 9 is a graph showing mean TFV plasma concentration (ng/mL) vs. time (0-24 hours) profiles in healthy subjects administered either a single dose of TDF or TAF/F.

### DETAILED DESCRIPTION

Provided herein are methods of treating a subject co-infected with a virus and tuberculosis (TB), comprising administering a therapeutically effective amount of tenofovir alafenamide (TAF) and a therapeutically effective amount of an antimycobacterial agent to the subject. In some embodiments, the TAF is administered once daily. In some embodiments, the TAF is administered twice daily.

In some embodiments of the methods provided herein, the subject is infected with a virus selected from hepatitis B virus (HBV), human papilloma virus (HPV), cytomegalovirus (CMV), Epstein-Barr virus (EBV), poliovirus, varicella zoster virus, coxsackie virus, and human immunodeficiency virus (HIV). In some embodiments, the subject is co-infected with TB and HIV. In some embodiments, the subject is co-infected with TB and HBV.

According to the present invention, the subject is administered an antimycobacterial agent that is effective against TB. Examples of suitable antimycobacterial agents include rifampin (rifampicin; RIF), rifabutin, rifapentine, isoniazid, ethambutol, pyrazinamide, dapsone, streptomycin, p-amino-salicylate, ethionamide, cycloserine, closerin, capreomycin, viomycin, enviomycin, amikacin, kanamycin, ciprofloxacin, levofloxacin, moxifloxacin, clofazamine, ethionamide, prothionamide, clarithromycin, linezolid, thioacetazone, thioridazine, bedaquiline, delamanid, pretomanid, and terizidone. In some embodiments, the antimycobacterial agent is RIF. In some embodiments, the antimycobacterial agent is one or more of RIF, isoniazid and/or pyrazinamide.

The antimycobacterial agent can be administered at any point during the day and need not be administered at the same time as TAF. In some embodiments, the antimycobacterial agent is administered once daily. In some embodiments, the antimycobacterial agent is administered on an empty stomach. In some embodiments, the antimycobacterial agent is coadministered with the dose of TAF. In some embodiments, where TAF is administered twice daily, the antimycobacterial agent is coadministered with one of the two daily doses of TAF. In further embodiments, the antimycobacterial agent is coadministered with the first daily dose of TAF. In some embodiments, the antimycobacterial agent is administered in a single dose of between about 100 mg and about 1000 mg, such as about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 800 mg, about 900 mg, or about 1000 mg. In some embodiments, the antimycobacterial agent is administered at a 600 mg daily dose. In some embodiments, the antimycobacterial agent is administered at a 10 mg/kg daily dose. In some embodiments, the antimycobacterial agent is RIF and is administered at a 10 mg/kg daily dose with the dose of TAF. In some embodiments, the antimycobacterial agent is RIF and is administered at a 600 mg daily dose with the dose of TAF. In some embodiments, where TAF is administered twice daily, the antimycobacterial agent is RIF and is administered at a 10 mg/kg daily dose together with the first daily dose of TAF. In some embodiments, where TAF is administered twice daily, the antimycobacterial agent is RIF and is administered at a 600 mg daily dose together with the first daily dose of TAF.

The amount of TAF that can be administered is generally sufficient to maintain therapeutic levels upon coadministration of the antimycobacterial agent. In some embodiments of the methods provided herein, the TAF is administered once daily at a dose of between about 5 mg and about 200 mg, such as about 5 mg, about 10 mg, about 25 mg, about 50 mg, about 75 mg, about 100 mg, about 125 mg, about 150 mg, or about 200 mg. In some embodiments, TAF is administered at a 25 mg dose once daily. In some embodiments, TAF is administered at a 10 mg dose once daily. In some embodiments of the methods provided herein, the TAF is administered twice daily (BID) at a dose of between about 5 mg and about 200 mg, such as about 5 mg, about 10 mg, about 25 mg, about 50 mg, about 75 mg, about 100 mg, about 125 mg, about 150 mg, or about 200 mg. In some embodiments, TAF is administered at a 25 mg dose twice daily, for a total daily dose of 50 mg. In some embodiments, TAF is administered at a 10 mg dose twice daily, for a total daily dose of 20 mg.

The methods of the invention can further comprise administering one or more therapeutic agents in addition to TAF and the antimycobacterial agent. In some embodiments, the additional therapeutic agents are selected from bictegravir, emtricitabine, elvitegravir, cobicistat, atazanavir, ritonavir, lopinavir, darunavir, rilpivirine, efavirenz, saquinavir, fosamprenavir and tipranavir. In some embodiments, the additional therapeutic agents are bictegravir and emtricitabine. In some embodiments, emtricitabine is administered in addition to the TAF and antimycobacterial agent. In some embodiments, both bictegravir and emtricitabine are administered in addition to the TAF and antimycobacterial agent. In some embodiments, elvitegravir, cobicistat, and emtricitabine are administered in addition to the TAF and antimycobacterial agent. In some embodiments, emtricitabine and rilpivirine are administered in addition to the TAF and antimycobacterial agent. In some embodiments, darunavir, cobicistat, and emtricitabine are administered in addition to the TAF and antimycobacterial agent.

In some embodiments, the method of treating a subject co-infected with a virus and tuberculosis (TB) does not include administration of an integrase inhibitor. For example, treatment according to the methods provided herein does not include administration of an integrase inhibitor such as bictegravir.

According to the methods provided herein, one or both of the daily doses of TAF can be coadministered with the one or more additional therapeutic agents. In some embodiments, one of the daily doses of TAF is coadministered with the one or more additional therapeutic agents. In some embodiments, both of the daily doses of TAF are coadministered with the one or more additional therapeutic agents. In some embodiments, the first daily dose of TAF is coadministered together with one or more additional therapeutics and the second daily dose of TAF is administered by itself.

In some embodiments, the TAF is formulated into a single tablet with the one or more additional therapeutic agents. In some embodiments, the single tablet is administered to the subject once daily. In some embodiments, the single tablet is administered to the subject twice daily. In some embodiments, the single tablet contains TAF, bictegravir, and emtricitabine. In some embodiments, the single tablet comprises 25 mg TAF, 50 mg bictegravir, and 200 mg emtricitabine. In some embodiments, the single tablet comprises TAF and emtricitabine (FTC). In some embodiments, the single tablet comprises 25 mg TAF and 200 mg emtricitabine (FTC). In some embodiments, the single tablet comprises 10 mg TAF and 200 mg emtricitabine (FTC).

In some embodiments, the single tablet comprises TAF, elvitegravir, cobicistat, and emtricitabine. In some embodiments, the single tablet comprises 10 mg TAF, 150 mg elvitegravir, 150 mg cobicistat, and 200 mg emtricitabine. In some embodiments, the single tablet comprises TAF, emtricitabine and rilpivirine. In some embodiments, the single tablet comprises 25 mg TAF, 200 mg emtricitabine and 25 mg rilpivirine. In some embodiments, the single tablet comprises TAF, darunavir, cobicistat, and emtricitabine. In some embodiments, the single tablet comprises 10 mg TAF, 800 mg darunavir, 150 mg cobicistat, and 200 mg emtricitabine.

In some embodiments, the present invention is directed to treating a subject co-infected with a virus and TB, comprising administering to the subject a therapeutically effective amount of (1) a single tablet twice daily comprising TAF, bictegravir, and emtricitabine and (2) a once daily dose of RIF. In some embodiments, the single tablet comprises 25 mg TAF, 50 mg bictegravir, and 200 mg emtricitabine. In some embodiments, RIF is administered at dose of 600 mg. In some embodiments, the virus is HIV.

In some embodiments, the present invention is directed to treating a subject co-infected with a virus and TB, comprising administering to the subject a therapeutically effective amount of (1) a single tablet once daily comprising TAF and emtricitabine and (2) a once daily dose of RIF. In some embodiments, the single tablet comprises 25 mg TAF and 200 mg emtricitabine. In some embodiments, RIF is administered at dose of 600 mg. In some embodiments, the virus is HIV.

The methods of the present invention can result in therapeutic PK profiles for simultaneous treatment of subjects co-infected with virus and TB, whereby the therapeutic effectiveness of TAF is maintained even when coadministered with an antimycobacterial agent. In some embodiments of the methods provided herein, following administration of TAF BID and the antimycobacterial agent, the daily TAF plasma exposure is not reduced by more than between about 15% and about 25%, such as not more than about 15%, about 20%, or about 25% of that measured for the same treatment, but wherein TAF is administered once daily in the absence of the antimycobacterial agent. In some embodiments, the TAF plasma exposure is not reduced by more than about 15%. In some embodiments, the TAF plasma exposure is not reduced by more than about 20%. In some embodiments, the TAF plasma exposure is not reduced by more than about 25%. In some embodiments, the antimycobacterial agent is RIF.

In some embodiments of the methods provided herein, following administration of TAF BID and the antimycobacterial agent, the daily TAF plasma AUC is between about 200 ng/mL and about 400 ng/mL, such as between about 250 ng/mL and about 350 ng/mL. In some embodiments, the TAF plasma exposure is about 250 ng/mL, about 260 ng/mL, about 270 ng/mL, about 280 ng/mL, about 290 ng/mL, about 300 ng/mL, about 310 ng/mL, about 320 ng/mL, about 330 ng/mL, about 340 ng/mL, or about 350 ng/mL. In some embodiments, the antimycobacterial agent is RIF.

In some embodiments of the methods provided herein, following administration of TAF BID and the antimycobacterial agent, the daily tenofovir (TFV) plasma exposure is not increased by more than between about 10% and about 20%, such as about 10%, or about 15%, or about 20% of that measured for the same treatment but wherein the TAF is administered once daily in the absence of the antimycobacterial agent. In some embodiments, the daily TFV plasma exposure is not more than measured for the same treatment but wherein the TAF is administered once daily in the absence of the antimycobacterial agent. In some embodiments, the antimycobacterial agent is RIF.

In some embodiments of the methods provided herein, following administration of TAF BID and the antimycobacterial agent, the daily TFV plasma AUC is between about 200 ng/mL and about 400 ng/mL, such as between about 250 ng/mL and about 350 ng/mL. In some embodiments, the TAF plasma exposure is about 250 ng/mL, about 260 ng/mL, about 270 ng/mL, about 280 ng/mL, about 290 ng/mL, about 300 ng/mL, about 310 ng/mL, about 320 ng/mL, about 330 ng/mL, about 340 ng/mL, or about 350 ng/mL. In some embodiments, the antimycobacterial agent is RIF.

In some embodiments of the methods provided herein, following administration of TAF BID and the antimycobacterial agent, the mean steady-state intracellular TFV-DP trough concentration is at least about 85 fmol/10⁶ cells, at least about 100 fmol/10⁶ cells, at least about 150 fmol/10⁶ cells, at least about 200 fmol/10⁶ cells, at least about 250 fmol/10⁶ cells, at least about 300 fmol/10⁶ cells, or at least about 350 fmol/10⁶ cells. In some embodiments, the antimycobacterial agent is RIF.

In some embodiments of the methods provided herein, following administration of TAF BID and the antimycobacterial agent, the daily TFV-DP intracellular exposure is not reduced by more than between about 20% and about 30%, such as about 20%, about 25%, or about 30% of that measured for the same treatment but wherein the TAF is administered once daily in the absence of the antimycobacterial agent. In some embodiments, the daily TFV-DP intracellular exposure is not reduced by more than about 20%. In some embodiments, the daily TFV-DP intracellular exposure is not reduced by more than about 25%. In some embodiments, the daily TFV-DP intracellular exposure is not reduced by more than about 30%. In some embodiments, the antimycobacterial agent is RIF.

In some embodiments, the treatment methods provided herein are bioequivalent with respect to TAF pharmacokinetic parameters when compared to the same treatment method but where TAF is administered once daily in the absence of an antimycobacterial agent. The FDA considers two products bioequivalent if the 90% CI of the relative mean Cₘₐₓ, AUC₍₀₋ₜ₎ and AUC_{(0-∞)} of the test (e.g., generic formulation) to reference (e.g., innovator brand formulation) is within 80% to 125% in the fasting state.

Also provided are methods of preventing co-infection with a virus in a subject infected with TB, comprising administering a therapeutically effective amount of TAF and a therapeutically effective amount of an antimycobacterial agent to the subject. In some embodiments, the TAF is administered once daily. In some embodiments, the TAF is administered twice daily. In some embodiments, the virus is selected from HBV, HPV, CMV, EBV, poliovirus, varicella zoster virus, coxsackie virus, and HIV. In some embodiments, the methods prevent co-infection with HIV in a subject already infected with TB. In some embodiments, the methods prevent co-infection with HBV in a subject already infected with TB.

As further described in the Examples, TAF BID plus RIF results in similar exposures as that observed following TAF QD (in the absence of RIF), which has demonstrated efficacy and safety. Thus, the methods provided herein can be used in patients co-infected with a virus and TB where treatment with once daily (QD) TAF is not recommended or expected to maintain efficacy in the presence of an antimycobacterial agent such as RIF.

As used herein, the term “subject” refers to an animal, preferably a mammal, including a human or non-human. The terms “patient” and “subject” may be used interchangeably herein.

As used herein, “co-infection” is the simultaneous infection of a patient by multiple pathogen species. Co-infection is of particular human health importance because pathogen species can interact within the host yielding a net effect often greater than the individual infections. A globally common co-infection involves tuberculosis and HIV.

As used herein, the term “coadminister” or “coadministration” refers to administration of two or more pharmaceutical agents within a certain time of each other such that their pharmaceutical effects on the subject overlap. Typically, coadministration means administration within 24 hours of each other, but in some embodiments, refers to the administration of two or more agents within 2 hours of each other, within 1 hour of each other, within 30 minutes of each other, or within 15 minutes of each other. In other embodiments, “coadminister” or “coadministration” refers to administration at the same time, either as part of a single formulation or as multiple formulations that are administered by the same or different routes.

As used herein, “therapeutically effective amount” refers to that amount of the compound being administered which will inhibit, reduce, alleviate, or eliminate the disease being treated, including the inhibition, reduction, alleviation, or elimination of one or more of the symptoms of the disease being treated. In some embodiments, “therapeutically effective amount” refers to a daily TAF plasma exposure after treatment with the methods provided herein that is not reduced by more than between about 15% and about 25% compared with a daily TAF plasma exposure measured for the same treatment but wherein the TAF is administered once daily in the absence of the antimycobacterial agent. In some embodiments, “therapeutically effective amount” refers to a daily TFV plasma exposure after treatment with the methods provided herein that is not reduced by more than between about 10% and about 20% compared with a daily TFV plasma exposure measured for the same treatment but wherein the TAF is administered once daily in the absence of the antimycobacterial agent. In some embodiments, “therapeutically effective amount” refers to a daily TFV plasma exposure after treatment with the methods provided herein that is the same as or less than that measured for the same treatment but wherein the TAF is administered once daily in the absence of the antimycobacterial agent. In some embodiments, “therapeutically effective amount” refers to a mean steady-state intracellular TFV-DP trough concentration after treatment with the methods provided herein that is between about 85 fmol/10⁶ cells and about 350 fmol/10⁶ cells. In some embodiments, “therapeutically effective amount” refers to a daily TFV-DP intracellular exposure after treatment with the methods provided herein that is not reduced by more than between about 20% and about 30% compared with a daily TFV-DP intracellular exposure measured for the same treatment but wherein the TAF is administered once daily in the absence of the antimycobacterial agent.

As used herein, “tenofovir alafenamide” or “TAF” each refer to the nucleoside analog reverse transcriptase inhibitor drug compound {9-[(R)-2-[[(S)-[[(S)-1-(isopropoxycarbonyl)ethyl]amino]phenoxyphosphinyl]-methoxy]propyl]adenine}. TAF may be associated with fumarate, such as monofumarate and hemifumarate salts or co-crystals (co-formers). See, e.g., U.S. Pat. Nos. 7,390,791, 7,803,788, and 8,754,065. It is understood that reference to “TAF” may be inclusive of a co-former and, in certain embodiments, associated with fumarate. TAF is marketed as Vemlidy® and is a component of the tablets Genvoya®, Descovy®, Odefsey®, and Symtuza®.

As used herein, “tenofovir disoproxil” or “TD” refers to the compound 9-[(R)-2-[[bis[[(isopropoxycarbonyl)oxy] methoxy]phosphinyl]methoxy]propyl]adenine. TD, a prodrug of tenoforvir may be associated with fumarate, such as monofumarate. See e.g. U.S. Pat. Nos. 5,922,695, 5,935,946, and 5,977,089. Tenofovir disoproxil fumarate is referred to as “TDF” and is marketed as Viread®.

As used herein, “tenofovir” or “TFV” refers to the compound (R)-9-(2-phosphonylmethoxypropyDadenine. TFV cannot be orally administered as a drug as it is a dianion at physiological pH and suffers from poor membrane permeability, as reflected in its poor in vitro anti-HIV activity in cell-based assays, and low oral bioavailability (Shaw et al. (1997) Pharm. Res. 14:1824-1829).

As used herein, “tenofovir diphosphate” or “TFV-DP” is a diphosphate derivative of tenofovir and is the active intracellular metabolite of TAF and TDF. TFV-DP is a potent inhibitor of HIV reverse transcriptase having long intracellular half-life measured to be 150 h in peripheral blood mononuclear cells (PBMC) isolated from patients (Hawkins et al. (2005) J. Acquir. Immune Defic. Syndr. 39:406-411; Pruvost et al. (2005) Antimicrob. Agents Chemother. 49:1907-1914).

As used herein, “rifampin” or “RIF” is an antimycobacterial agent, marketed as RIFADIN®, that inhibits bacterial DNA-dependent RNA synthesis, and is indicated in the treatment of all forms of tuberculosis as part of a multicomponent antibacterial regimen. Rifampin is a substrate of P-gp and SLCO1B1 and a strong inducer of CYP1A2, CYP2A6, CYP2B6, CYP2C19, CYP2C8, CYP3A4 and P-gp.

As used herein, “bictegravir” or “BIC” refers to the integrase inhibitor (2R,5S,13aR)-7,9-dioxo-10-[(2,4,6-trifluorobenzyl carbamoyl]-2,3,4,5,7,9,13,13a-octahydro-2,5-methanopyrido[1′,2′:4,5]pyrazino[2,1-b][1,3]oxazepin-8-olate. BIC may be a pharmaceutically acceptable salt, such as a sodium salt, or associated with co-crystals (co-formers). It is understood that reference to “BIC” may be inclusive of a co-former, such as sodium.

Any of the compounds described herein can be in the free base form or as a pharmaceutically acceptable salt thereof.

The pharmaceutical agents of the methods of the invention can be administered in the form of pharmaceutical compositions, such as the combination of the pharmaceutical agent(s) and a carrier or other excipients. Pharmaceutical compositions can be prepared in a manner well known in the pharmaceutical art, and can be administered by a variety of routes, depending upon whether local or systemic treatment is desired and upon the area to be treated. In some embodiments, the pharmaceutical agents of the methods of the invention are administered orally, such as in the form of a tablet or capsule.

As used herein, the term “AUC” refers to the area under the drug concentration-time curve. The term “AUC₀₋ₜ,” as used herein, refers to the area under the drug concentration-time curve from t=0 to the last measurable concentration. For example, “AUC₀₋₂₄” refers to the area under the drug concentration-time curve from t=0 to t=24 hours.

As used herein, the term “Cₘₐₓ,” refers to the maximum observed plasma concentration following administration of a drug.

“Cₜₐᵤ” or “C_{trough}” is a pharmacokinetic (PK) parameter that refers to the concentration of the drug at the end of the dosing interval. This parameter is obtained by direct measurement of the drug concentrations in a plasma sample collected from the study subject at the end of the dosing interval (e.g., 24 hours post-dose) using a validated liquid chromatography/tandem mass spectrometry (LC/MS/MS) bioanalytical assay.

As used herein, the term “coefficient of variation (CV)” refers to the ratio of the sample standard deviation to the sample mean. It is often expressed as a percentage.

As used herein, the term “confidence interval (CI),” refers to a range of values which will include the true average value of a parameter a specified percentage of the time.

As used herein, “C_{trough}^{”} is a pharmacokinetic (PK) parameter that refers to the concentration of the drug at the end of the dosing interval. This parameter is obtained by direct measurement of the drug concentrations in a plasma sample collected from the study subject at the end of the dosing interval (e.g., 24 hours post-dose) using a validated liquid chromatography/tandem mass spectrometry (LC/MS/MS) bioanalytical assay.

The methods of the invention can include the administration of additional therapeutic agents for the prevention or treatment of viral infections and other diseases. Certain examples are listed below.

### HIV Combination Therapy

In some embodiments of the methods provided herein, the additional therapeutic agent may be an anti-HIV agent. In some embodiments, the additional therapeutic agent is selected from among HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T), latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitors, HIV Vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

### HIV Combination Drugs

Examples of combination drugs include ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir, emtricitabine, tenofovir alafenamide); darunavir, tenofovir alafenamide, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine; Vacc-4x and romidepsin; and APH-0812; bictegravir, emtricitabine, tenofovir alafenamide hemifumarate; bictegravir sodium, emtricitabine, tenofovir alafenamide hemifumarate; bictegravir, lamivudine, abacavir; bictegravir sodium, lamivudine, abacavir sulfate.

### Other HIV Drugs

Examples of other drugs for treating HIV include acemannan, alisporivir, BanLec, deferiprone, Gamimune, metenkefalin, naltrexone, Prolastin, REP 9, RPI-MN, VSSP, Hlviral, SB-728-T, 1,5-dicaffeoylquinic acid, rHIV7-shl-TAR-CCRSRZ, AAV-eCD4-Ig gene therapy, MazF gene therapy, BlockAide, ABX-464, AG-1105, APH-0812, BIT-225, CYT-107, HGTV-43, HPH-116, HS-10234, IMO-3100, IND-02, MK-1376, MK-8507, MK-8591, NOV-205, PA-1050040 (PA-040), PGN-007, SCY-635, SB-9200, SCB-719, TR-452, TEV-90110, TEV-90112, TEV-90111, TEV-90113, RN-18, Immuglo, and VIR-576.

### HIV Protease Inhibitors

Examples of HIV protease inhibitors include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, and TMC-310911.

### HIV Reverse Transcriptase Inhibitors

Examples of HIV non-nucleoside or non-nucleotide inhibitors of reverse transcriptase include dapivirine, delavirdine, delavirdine mesylate, doravirine, efavirenz, etravirine, lentinan, nevirapine, rilpivirine, ACC-007, AIC-292, KM-023, PC-1005, and VM-1500.

Examples of HIV nucleoside or nucleotide inhibitors of reverse transcriptase include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, GS-9131, GS-9148, MK-8504 and KP-1461.

### HIV Integrase Inhibitors

Examples of HIV integrase inhibitors include elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169 and cabotegravir.

Examples of HIV non-catalytic site, or allosteric, integrase inhibitors (NCINI) include CX-05045, CX-05168, and CX-14442.

### HIV Entry Inhibitors

Examples of HIV entry (fusion) inhibitors include cenicriviroc, CCR5 inhibitors, gp41 inhibitors, CD4 attachment inhibitors, gp120 inhibitors, and CXCR4 inhibitors.

Examples of CCR5 inhibitors include aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

Examples of gp41 inhibitors include albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

Examples of CD4 attachment inhibitors include ibalizumab and CADA analogs Examples of gp120 inhibitors include Radha-108 (receptol) 3B3-PE38, BanLec, bentonite-based nanomedicine, fostemsavir tromethamine, IQP-0831, and BMS-663068

Examples of CXCR4 inhibitors include plerixafor, ALT-1188, N15 peptide, and vMIP (Haimipu).

### HIV Maturation Inhibitors

Examples of HIV maturation inhibitors include BMS-955176 and GSK-2838232.

### Latency Reversing Agents

Examples of latency reversing agents include histone deacetylase (HDAC) inhibitors, proteasome inhibitors such as velcade, protein kinase C (PKC) activators, Smyd2 inhibitors, BET-bromodomain 4 (BRD4) inhibitors, ionomycin, PMA, SAHA (suberanilohydroxamic acid, or suberoyl, anilide, and hydroxamic acid), AM-0015, ALT-803, NIZ-985, NKTR-255, IL-15 modulating antibodies, JQ1, disulfiram, amphotericin B, and ubiquitin inhibitors such as largazole analogs, and GSK-343.

Examples of HDAC inhibitors include romidepsin, vorinostat, and panobinostat.

Examples of PKC activators include indolactam, prostratin, ingenol B, and DAG-lactones.

### Capsid Inhibitors

Examples of capsid inhibitors include capsid polymerization inhibitors or capsid disrupting compounds, HIV nucleocapsid p7 (NCp7) inhibitors such as azodicarbonamide, HIV p24 capsid protein inhibitors, AVI-621, AVI-101, AVI-201, AVI-301, and AVI-CAN1-15 series.

### Immune-Based Therapies

Examples of immune-based therapies include toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 modulators; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Fc fusion protein, normferon, peginterferon alfa-2a, peginterferon alfa-2b, recombinant interleukin-15, RPI-MN, GS-9620, STING modulators, RIG-I modulators, NOD2 modulators, and IR-103.

Phosphatidylinositol 3-kinase (PI3K) Inhibitors

Examples of PI3K inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of integrin alpha-4/beta-7 antagonists include PTG-100, TRK-170, abrilumab, etrolizumab, carotegrast methyl, and vedolizumab.

### HIV Antibodies, Bispecific Antibodies, and “Antibody-Like” Therapeutic Proteins

Examples of HIV antibodies, bispecific antibodies, and “antibody-like” therapeutic proteins include DARTs®, DUOBODIES®, BITES®, XmAbs®, TandAbs®, Fab derivatives, bnABs (broadly neutralizing HIV-1 antibodies), BMS-936559, TMB-360, and those targeting HIV gp120 or gp41, antibody-Recruiting Molecules targeting HIV, anti-CD63 monoclonal antibodies, anti-GB virus C antibodies, anti-GP120/CD4, CCR5 bispecific antibodies, anti-nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), ibalizumab, Immuglo, and MB-66.

Examples of those targeting HIV in such a manner include bavituximab, UB-421, C2F5, 2G12, C4E10, C2F5+C2G12+C4E10, 8ANC195, 3BNC117, 3BNC60, 10-1074, PGT145, PGT121, PGT-151, PGT-133, MDX010 (ipilimumab), DH511, N6, VRC01, PGDM1400, A32, 7B2, 10E8, 10E8v4, CAP256-VRC26.25, DRVIA7, VRC-07-523, VRC-HIVMAB080-00-AB, VRC-HIVMAB060-00-AB, MGD-014 and VRC07. An example of an HIV bispecific antibody includes MGD014.

### Pharmacokinetic Enhancers

Examples of pharmacokinetic enhancers include cobicistat and ritonavir.

### Additional Therapeutic Agents

Examples of additional therapeutic agents include the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include peptide vaccines, recombinant subunit protein vaccines, live vector vaccines, DNA vaccines, CD4-derived peptide vaccines, vaccine combinations, rgp120 (AIDSVAX), ALVAC HIV (vCP1521)/AIDSVAX B/E (gp120) (RV144), monomeric gp120 HIV-1 subtype C vaccine, Remune, ITV-1, Contre Vir, Ad5-ENVA-48, DCVax-001 (CDX-2401), Vacc-4x, Vacc-05, VAC-3S, multiclade DNA recombinant adenovirus-5 (rAd5), Pennvax-G, Pennvax-GP, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), gp140[delta]V2.TV1+MF-59, rVSVIN HIV-1 gag vaccine, SeV-Gag vaccine, AT-20, DNK-4, ad35-Grin/ENV, TBC-M4, HIVAX, HIVAX-2, NYVAC-HIV-PT1, NYVAC-HIV-PT4, DNA-HIV-PT123, rAAV1-PG9DP, GOVX-B11, GOVX-B21, TVI-HIV-1, Ad-4 (Ad4-env Clade C+Ad4-mGag), EN41-UGR7C, EN41-FPA2, PreVaxTat, AE-H, MYM-V101, CombiHlVvac, ADVAX, MYM-V201, MVA-CMDR, DNA-Ad5 gag/pol/nef/nev (HVTN505), MVATG-17401, ETV-01, CDX-1401, rcAD26.MOS1.HIV-Env, Ad26.Mod.HIV vaccine, AGS-004, AVX-101, AVX-201, PEP-6409, SAV-001, ThV-01, TL-01, TUTI-16, VGX-3300, IHV-001, and virus-like particle vaccines such as pseudovirion vaccine, CombiVlCHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines, gag-based DNA vaccine, GI-2010, gp41 HIV-1 vaccine, HIV vaccine (PIKA adjuvant), I i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, recombinant peptide vaccine (HIV infection), NCI, rgp160 HIV vaccine, RNActive HIV vaccine, SCB-703, Tat Oyi vaccine, TBC-M4, therapeutic HIV vaccine, UBI HIV gp120, Vacc-4x+romidepsin, variant gp120 polypeptide vaccine, rAd5 gag-pol env A/B/C vaccine, DNA.HTI and MVA.HTI.

### HIV Combination Therapy

In a particular embodiment, a compound disclosed herein, or a pharmaceutically acceptable salt thereof, is combined with one, two, three, four or more additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

### Birth Control (Contraceptive) Combination Therapy

Therapeutic agents used for birth control (contraceptive) include cyproterone acetate, desogestrel, dienogest, drospirenone, estradiol valerate, ethinyl estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

Gene therapy and cell therapy includes the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; and genetic approaches to modify cellular activity to further alter endogenous immune responsiveness against the infection.

Examples of dendritic cell therapy include AGS-004.

### Gene Editors

The genome editing system is selected from the group consisting of: a CRISPR/Cas9 system, a zinc finger nuclease system, a TALEN system, a homing endonucleases system, and a meganuclease system.

Examples of HIV targeting CRISPR/Cas9 systems include EBT101.

### CAR-T Cell Therapy

CAR-T cell therapy includes a population of immune effector cells engineered to express a chimeric antigen receptor (CAR), wherein the CAR comprises an HIV antigen-binding domain. The HIV antigen includes an HIV envelope protein or a portion thereof, gp120 or a portion thereof, a CD4 binding site on gp120, the CD4-induced binding site on gp120, N glycan on gp120, the V2 of gp120, and the membrane proximal region on gp41. The immune effector cell is a T cell or an NK cell. In some embodiments, the T cell is a CD4+ T cell, a CD8+ T cell, or a combination thereof.

An examples of HIV CAR-T includes VC-CAR-T.

### TCR-T Cell Therapy

TCR-T cells are engineered to target HIV derived peptides present on the surface of virus-infected cells.

It will be appreciated by one of skill in the art that the additional therapeutic agents listed above may be included in more than one of the classes listed above. The particular classes are not intended to limit the functionality of those compounds listed in those classes.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

In the following description of the examples, specific embodiments in which the invention may be practiced are described. These embodiments are described in sufficient detail to enable those skilled in the art to practice the invention. Other embodiments may be utilized, and logical and other changes may be made without departing from the scope of the invention. The following detailed description is, therefore, not to be taken in a limiting sense, and the scope of the invention is defined only by the appended claims, along with the full scope of equivalents to which such claims are entitled

### EXAMPLES

### Example 1: Phase 1 Study to Evaluate Pharmacokinetics Following Administration of Bictegravir/Emtricitabine/Tenofovir Alafenamide (B/F/TAF) Twice Daily with Rifampin in Healthy Human Subjects

This study evaluated the steady state pharmacokinetics of TAF, TAF metabolite TFV, and emtricitabine (FTC) following coadministration of bictegravir/emtricitabine/tenofovir alafenamide (B/F/TAF) fixed dose combination (FDC) twice daily with rifampin 600 mg once daily compared to those of B/F/TAF FDC once daily administered alone.

### Study Design

In this Phase 1, open-label, parallel multiple dose study, healthy volunteers were assigned to one of two cohorts (cohort 1 (n=26) or cohort 2 (n=26)) and received study treatments beginning on day 1 and continuing for 28 days (cohort 1) or 35 days (cohort 2). The enrollment and demographics of the volunteers is shown in Table 1.

TABLE 1
Enrollment and demographics

| | Cohort 1 | | Cohort 2 | |
|---|---|---|---|---|
| | (B/F/TAF QD) | | (B/F/TAF BID + RIF) | |
| Mean age, y (SD) | 35 | (7.1) | 36 | (5.8) |
| Female sex, n (%) | 13 | (50) | 13 | (50) |
| Race, n (%) | | | | |
| Black | 6 | (23.1) | 6 | (23.1) |
| White | 20 | (76.9) | 20 | (76.9) |
| Hispanic or Latino | 26 | (100) | 26 | (100) |
| Mean BMI, kg/m² (SD) | 26.2 | (2.26) | 26.0 | (2.06) |
| Mean eGFR, mL/min (SD) | 129.18 | (24.919) | 129.77 | (22.992) |

The duration of the study was approximately 57 days for cohort 1 and approximately 44 days for cohort 2. The treatments were as follows:
Treatment A: Once daily B/F/TAF (50/200/25 mg) FDC, 1 tablet administered orally 2 hours after a standard breakfast meal in the morning.
Treatment B: Twice daily B/F/TAF (50/200/25 mg) FDC and single dose rifampin (RIF) 600 mg (2×300 mg capsules) coadministered in the morning.
   Morning dose: 1 tablet B/F/TAF (50/200/25 mg) FDC and 2 capsules RIF 300 mg coadministered 2 hours after a standard breakfast.
   Evening dose: 1 tablet B/F/TAF (50/200/25 mg) FDC administered 2 hours after a standard dinner. Evening dose was administered approximately 12 hours after the morning dose.
   No evening dose of B/F/TAF (50/200/25 mg) FDC was given on Days 1 and 28.
Treatment C: Once daily RIF 600 mg (2×300 mg capsules), administered orally 2 hours after a standard breakfast in the morning.

The study procedures and frequency are shown in Tables 2 and 3.

TABLE 2
Cohort 1 study procedure

Cohort 1

| | | | 35 (±2), 42 (±2), |
|---|---|---|---|
| Study Days | 1-28 | 29 | 49 (±2), and 56 (±2) |
| Treatment | A | Discharge | PK washout |
| | | | Follow-up visits |

TABLE 3
Cohort 2 study procedure

Cohort 2

| Study Days | 1-28 | 29-35 | 36 | 43 |
|---|---|---|---|---|
| Treatment | B | C | Discharge | Follow-up |
| | | | | phone call |

Pharmacokinetic assessments of plasma PK and PBMC PK were evaluated. The plasma PK parameters of BIC, TAF, TFV and FTC were calculated as applicable with and without RIF and included AUC₀₋ₗₐₛₜ, AUCₜₐᵤ, Cₘₐₓ, Cₜₐᵤ, Cₗₐₛₜ, Tₘₐₓ, Tₗₐₛₜ, CL/F, V_{z}/F, and t_{1/2}. PK parameters of TFV-DP and FTC-TP in PBMCs were calculated as applicable and included AUCₜₐᵤ, C_{trough}, Cₘₐₓ, accumulation ratios, and t_{1/2} with and without RIF.

For plasma PK, serial blood samples were collected on days 1 and 28 in both cohorts at times relative to the morning dose of study drug B/F/TAF as follows.

Day 1:
0 (pre-dose≤5 minutes).
0.25, 0.5, 0.75, 1, 1.5, 2, 3, 4, 6, 8, 10, 12, 16 and 24 hours post-dose.

Plasma concentrations of TAF and TFV were determined and pharmacokinetics were evaluated as applicable.

Day 28:
0 (pre-dose≤5 minutes).
0.25, 0.5, 0.75, 1, 1.5, 2, 3, 4, 6, 8, 10, 12, 16 and 24 hours post-dose.

Plasma concentrations of BIC, TAF, FTC, and TFV were determined and pharmacokinetics were evaluated as applicable.

For peripheral blood mononuclear cells (PBMC) PK, blood samples were collected at the following time points relative to the morning dose of study drug B/F/TAF as follows.

Cohort 1:
Days 1 and 28: 0 (pre-dose), 1, 2, 3, 4, 6, 8, 12, 16, 20 and 24 hours post-dose.
Trough PBMC samples prior to the morning dose of B/F/TAF were collected on Days 11, 14, 17, 20, 22, 24 and 26. PBMC samples were collected on Days 35 (±2), 42 (±2), 49 (±2), and 56 (±2) during follow-up visits.

Cohort 2:
Day 1: 0 (pre-dose), 1, 2, 3, 4, 6, 8 and 12 hours post-dose.
Day 28: 0 (pre-dose), 1, 2, 3, 4, 6, 8, 12, 16, 20 and 24 hours post-dose.
Trough PBMC samples prior to the morning dose of B/F/TAF were collected on Days 11, 14, 17, 20, 22, 24 and 26. PBMC samples on Days 30, 32, 34 and 36 were collected pre-dose of RIF dosing. PBMC concentrations of TFV-DP and FTC-TP were determined and pharmacokinetics evaluated as applicable.

Plasma concentrations were determined using validated liquid chromatography-tandem mass spectrometry (LC/MS/MS) assays. Intracellular PBMC TFV-DP concentrations were determined via a validated LC/MS/MS assay combined with PBMC cell counting via a DNA quantitation procedure. Geometric least-squares mean (GLSM) ratios and associated 90% confidence intervals (CI) were used for statistical comparisons of exposures.

### Results

Pharmacokinetics

TAF

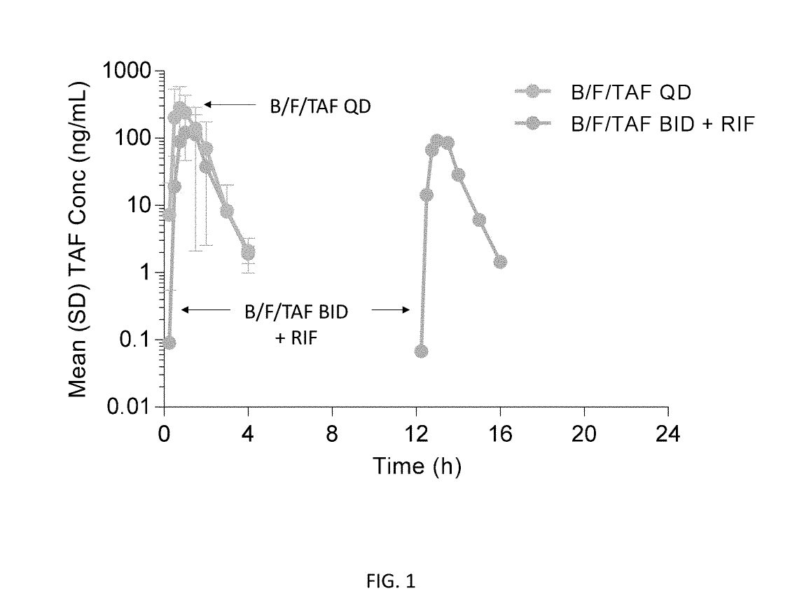

The pharmacokinetics of TAF were evaluated following B/F/TAF BID+RIF and compared to B/F/TAF QD. Results are shown in Table 4 and FIG. 1.

TABLE 4
Daily TAF plasma exposure

| | | Cohort 2 | |
|---|---|---|---|
| TAF PK | Cohort 1 | B/F/TAF | GLSM ratio |
| Mean (% CV) | B/F/TAF QD | BID + RIF | 90% CI |
| AUC₀₋₂₄ (ng · h/mL) | 345 (52) | 290 (48) | 85.8 (69.7, 106) |
| Cₘₐₓ (ng/mL) | 471 (72) | 207 (64) | 44.1 (33.2, 58.7) |

The results show that TAF plasma exposure in subjects that received B/F/TAF BID+RIF treatment over a 24 hour period was decreased by about 15% as compared to TAF plasma exposure in subjects that received B/F/TAF QD treatment.

TFV

The pharmacokinetics of TFV were also evaluated 24 hours following either B/F/TAF BID+RIF treatment or B/F/TAF QD treatment. As shown in Table 5, the total daily tenofovir (TFV) plasma exposure in subjects that received B/F/TAF BID+RIF treatment over a 24 hour period was decreased by about 20% as compared to TFV plasma exposure in subjects that received B/F/TAF QD treatment without RIF.

TABLE 5
Daily TFV plasma exposure

| | | Cohort 2 | |
|---|---|---|---|
| TFV PK | Cohort 1 | B/F/TAF | GLSM ratio |
| Mean (% CV) | B/F/TAF QD | BID + RIF | 90% CI |
| AUC₀₋₂₄ (ng · h/mL) | 348 (20) | 277 (19) | 79.9 (73.1, 87.3) |

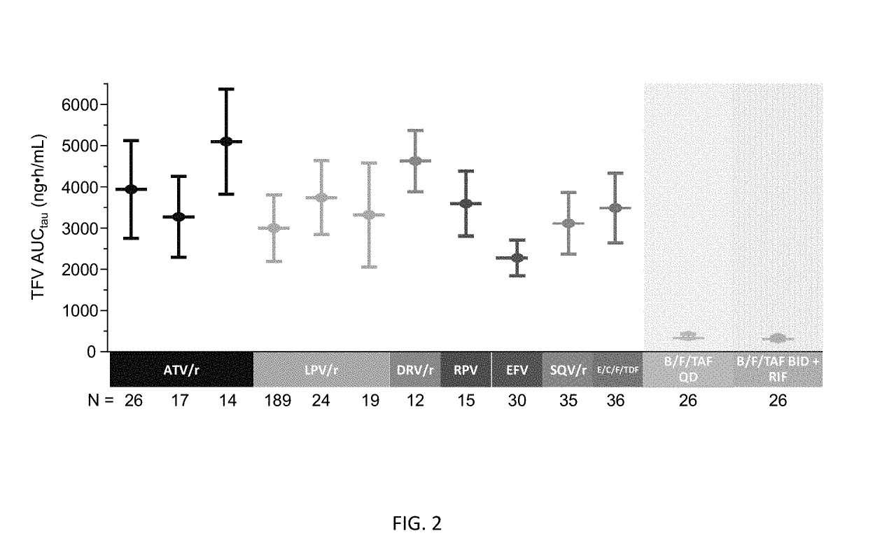

The data was also compared to prior PK results from TDF-containing treatment regimens which included concomitant treatment with atazanavir/ritonavir (ARV/r); lopinavir/ritonavir (LPV/r); darunavir/ritonavir (DRV/r); rilpivirine (RPV); efavirenz (EFV); saquinavir/ritonavir (SQV/r); and elvitegravir/cobicistat/emtricitabine/TDF (E/C/F/TDF). Despite BID TAF administration, the total overall systemic TFV plasma exposure over 24 hours was markedly lower than the TDF-containing regimens (FIG. 2).

TFV-DP

The pharmacokinetics of TFV-DP were evaluated 24 hours following either B/F/TAF BID+RIF treatment or B/F/TAF QD treatment without RIF. As shown in Table 6, the total daily TFV-DP intracellular exposure in subjects that received B/F/TAF BID+RIF treatment over a 24 hour period was decreased by about 24% as compared to TFV-DP intracellular exposure in subjects that received B/F/TAF QD treatment without RIF.

TABLE 6
Intracellular TFV-DP exposure

| TFV PK | GLSM ratio (90% CI) |
|---|---|
| AUC₀₋₂₄ (fmol · h/million cells) | 76.3 (58.7, 99.2) |

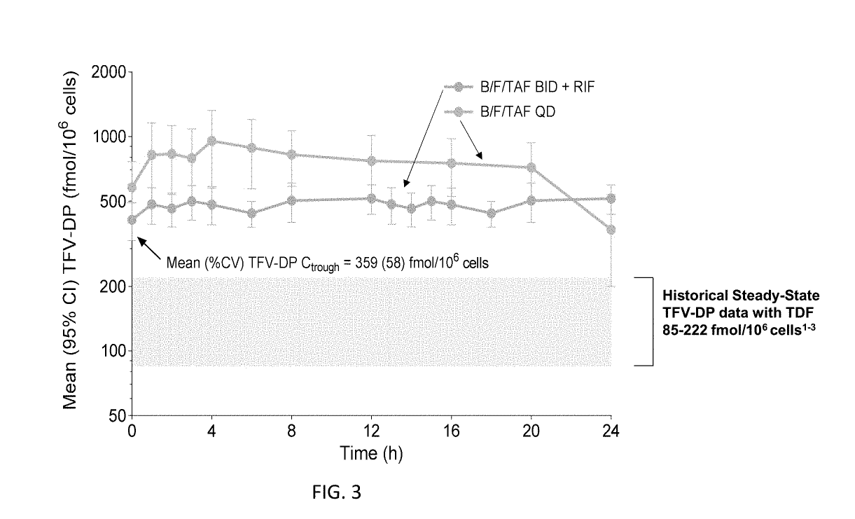

As shown in FIG. 3, B/F/TAF BID+RIF treatment resulted in a mean (% CV) steady-state intracellular TFV-DP trough concentration of 359 (58) fmol/10⁶ cells. Historical steady-state TFV-DP data with TDF has shown a threshold effectiveness concentration of between 85-222 fmol/10⁶ cells (Pruvost et al. (2005) Antimicrob. Agents Chemother. 49:1907-1914; Damond (2010) JCM; Hawkins et al. (2005) J. Acquir. Immune Defic. Syndr. 39:406-411). Thus, administration of B/F/TAF BID+RIF, results in a mean (% CV) TFV-DP trough concentration that is higher than the threshold level of effectiveness achieved when using TDF.

### Example 2: Evaluation of Drug-Drug Interactions Between Bictegravir and Rifampin

This study evaluated the effects of rifampin (RIF) on the pharmacokinetics of bictegravir (BIC; GS-9883) in healthy human subjects.

### Study Design

This was a Phase 1, open-label, multiple-dose, multiple-cohort, adaptive design study in healthy adult subjects. Eligible subjects received treatment with either a single dose of 75 mg BIC or a regimen that included both BIC and RIF. The treatment regimens were as follows and as shown in Table 7 below.
Treatment A (Reference): Single dose of BIC 75 mg administered orally in the morning under fed conditions.
Treatment C: Once daily doses of RIF 600 mg administered orally in the morning under fed conditions
Treatment A+C (Test): Single dose of BIC 75 mg (3×25-mg tablets) coadministered (within 5 minutes) with RIF 600 mg (2×300-mg capsules) orally in the morning under fed conditions.

TABLE 7
Study procedure

| Day 1 | Days 2-4 | Days 5-14 | Day 15 | Days 16-18 |
|---|---|---|---|---|
| A | Washout | C | A + C | C |

The duration of dosing was 9 days (with a 3-day washout between the first and second doses of study treatment). The study duration (not including screening) was approximately 21 days. The study had n=15 subjects.

The following single-dose plasma PK parameters of BIC were calculated, as applicable: AUCₗₐₛₜ, AUC_{inf}, % AUCₑₓₚ, Cₘₐₓ, Cₗₐₛₜ, Tₘₐₓ, Tₗₐₛₜ, λ_{z}, CL/F, V_{z}/F, and t_{1/2}. In addition, the following multiple-dose plasma PK parameters of GS-9883 were calculated, as applicable: Cₜₐᵤ, Cₘₐₓ, Cₗₐₛₜ, AUCₜₐᵤ, Tₘₐₓ, Tₗₐₛₜ, t_{1/2}, and CL/F.

Serial blood samples for intensive PK sampling were collected at the following time points relative to the morning dose of study drug(s) administered: 0 (pre-dose) and 0.5, 1, 1.5, 2, 3, 4, 6, 8, 10, 12, 16, 24, 36, 48, 72, and 96 hours post-dose on Days 1 and 15. Daily pre-dose PK sampling was collected at days 10 and 14.

Plasma concentrations and PK parameters for BIC were listed and summarized using descriptive statistics by treatment. An analysis of variance (ANOVA) was performed for the natural logarithms of the following PK parameters for BIC: AUC_{inf}, AUCₗₐₛₜ, and Cₘₐₓ for single dose and AUCₜₐᵤ, Cₜₐᵤ, and Cₘₐₓ for multiple doses. The ANOVA model included treatment as a fixed effect and subject as a random effect. Geometric least square mean (GLSM) ratios between test treatment (BIC+RIF) and reference treatment (BIC alone) and 90% confidence intervals (CIs) were constructed.

### Pharmacokinetics Results

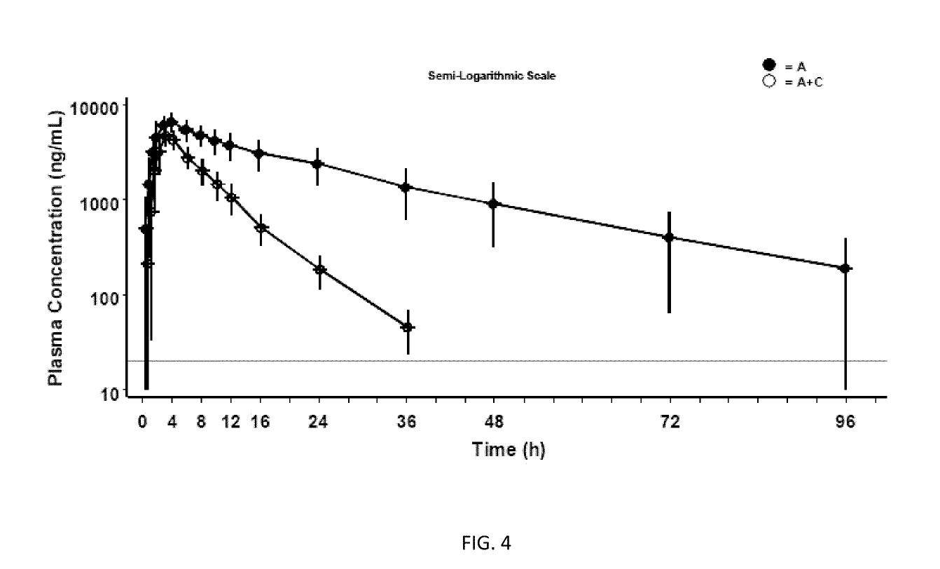

The mean (SD) BIC plasma concentrations over time following administration of a single dose of BIC 75 mg alone (A, Day 1) or with RIF 600 mg (A+C, Day 15) were determined (FIG. 4). Table 8 summarizes single-dose BIC PK parameters following administration alone or with RIF under fed conditions.

TABLE 8
Pharmacokinetic results

| | Mean (% CV) | | |
|---|---|---|---|
| BIC PK | | Reference | Test |
| parameter | | BIC | BIC + RIF |

| | | | | |
|---|---|---|---|---|
| AUC_{inf} (ng · h/mL) | 155,986.3 | (41.8) | 36,398.0 | (21.5) |
| % AUCₑₓₚ (%) | 3.1 | (83.8) | 1.1 | (51.4) |
| AUCₗₐₛₜ (h*ng/mL) | 149,814.3 | (38.5) | 36,014.8 | (21.8) |
| Cₘₐₓ (ng/mL) | 7118.7 | (17.0) | 5131.3 | (15.7) |
| Cₗₐₛₜ (ng/mL) | 192.7 | (103.2) | 45.7 | (41.3) |
| Tₘₐₓ (h) | 3.00 | (3.00, 4.00) | 3.00 | (3.00, 4.00) |
| Tₗₐₛₜ (h) | 95.93 | (95.93, 95.93) | 36.00 | (36.00, 36.00) |
| t_{1/2} (h) | 18.09 | (14.47, 20.75) | 5.65 | (5.30, 6.18) |
| V_{z}/F (mL) | 13,351.9 | (18.3) | 17,493.6 | (18.4) |
| CL/F (mL/h) | 545.1 | (32.9) | 2131.1 | (17.0) |

Decreases in BIC exposure (AUC and Cₘₐₓ) were observed when BIC was coadministered with RIF. Median BIC Tₘₐₓ was the same (3 hours) for both treatments. Median t_{1/2} was approximately 18.1 hours for BIC alone and decreased to approximately 5.7 hours for BIC with RIF.

Table 9 shows a statistical comparison between BIC coadministered with RIF and BIC administered alone for the BIC PK parameters AUCₗₐₛₜ, AUC_{inf}, and Cₘₐₓ.

TABLE 9
Statistical comparison of BIC plasma PK parameters

| | GLSM | | % GLSM ratio |
|---|---|---|---|
| | Reference | Test | (90% CI) |
| BIC PK parameter | BIC | BIC + RIF | (Test/Reference) |
| AUCₗₐₛₜ (h*ng/mL) | 141,267.52 | 35,347.97 | 25.02 (22.71, 27.57) |
| AUC_{inf} (ng · h/mL) | 145,765.08 | 35,742.97 | 24.52 (22.00, 27.33) |
| Cₘₐₓ (ng/mL) | 7023.57 | 5071.53 | 72.21 (67.06, 77.75) |

Co-administration of a single-dose of 75 mg BIC with 600 mg RIF under fed conditions resulted in decreases of 75% in AUC_{inf} and 28% in Cₘₐₓ relative to those for a single dose of 75 mg BIC alone; there was a 69% decrease in median t_{1/2}, indicating enhancement of clearance by RIF.

### Example 3: Phase 1 Study to Evaluate the Effect of Rifampin (RIF) on Plasma Pharmacokinetics of Emtricitabine (FTC) and Tenofovir Alafenamide (TAF) and Intracellular Tenofovir Diphosphate (TFV-DP) and Emtricitabine Triphosphate (FTC-TP)

This study assessed the pharmacokinetics of TAF, plasma tenofovir, intracellular tenofovir-diphosphate (TFV-DP), emtricitabine, and emtricitabine triphosphate (FTC-TP), during administration of TAF/FTC and TDF alone and during co-administration of TAF/FTC or TDF plus rifampin in HIV-negative healthy volunteers. This study also investigated the association between genetic polymorphisms in drug disposition genes and drug exposure and the impact of anti-retroviral drugs on platelet function in people living with HIV.

### Study Design

In this Phase 1, non-randomized study, healthy volunteers (n=21) received all study medications in the same order for a total of 84 days. The participant and the study doctor knew which study medications the participant was taking at all times during the study. The enrollment and demographics of the volunteers is shown in Table 10.

TABLE 10
Enrollment and demographics

| | | |
|---|---|---|
| Mean age, years (range) | 33 | (22-58) |
| Male sex, n (%) | 7 | (33) |
| Ethnicity, n (%) | | |
| Black | 7 | (33) |
| White | 11 | (53) |
| Others | 3 | (14) |
| Mean BMI, kg/m² (range) | 25 | (19-35) |

The total duration of the participant's involvement in the study was 85 days plus a screening visit up to 28 days prior to the start of the study, and a follow up visit 27 to 35 days after the last blood measurement.

The participants visited the clinic on 18 occasions, of which three visits involved staying in the unit for approximately 12 hours (days 28, 56 and 84). Blood samples were taken from the participants throughout the study to measure the levels of TAF/FTC, TDF and RIF in the blood. Blood and urine were also collected throughout the study for safety analysis to ensure the participants were healthy to take part or to continue taking part in the study. The total amount of blood collected from each participant during the study was approximately 590 mL (approximately a pint).

All participants were administered the drugs as follows:
Phase 1: TAF/FTC (Descovy®) 25/200 mg once daily for 28 days (Days 1-28).
Phase 2: TAF/FTC (Descovy®) 25/200 mg once daily plus RIF (Rifadin®) 600 mg once daily for 28 days (Days 29-56).
Phase 3: TDF (Viread®) 245 mg once daily for 28 days (Days 57-84).

Participants were dosed in the clinic on Days 1, 7, 14, 21, 28, 29, 35, 42, 49, 56, 57, 63, 70, 77, 84 and 85. On all others days, participants were required to take the dose at home.

In Phases 1 and 3, TDF and TAF/FTC dosing was administered following a standard breakfast, along with 240 mL of water. Participants then took the study drug at home at the same time every day within 15 minutes after a standard breakfast.

In Phase 2, the RIF dose (either taken in the unit or at home) was taken on an empty stomach. Then, at least 30 mins after the RIF dose, participants had a standard meal followed by the TAF/FTC dose.

Intensive pharmacokinetic visits occurred on days 28, 56, and 84. Participants were admitted to the unit in the morning on Days 28, 56 and 84 and remained in the unit for approximately 12 hours. They fasted for 8 hours overnight prior to attending. Blood samples were collected for:
Serial blood sample collection for plasma drug concentration at the following time points: pre-dose (within 10 minutes before dosing), 2, 4, 6, 8, 12 hr post dose (all ±5 minutes).
Serial blood sample collection for peripheral blood mononuclear cell (PBMC) drug concentration at the following time points: pre-dose (within 10 minutes before dosing), 2, 8 and 12 hr post dose (all ±5 minutes).
On Days 28 and 84 only: Platelet function investigation. Patients were able to leave the unit after the 12-hour sample to return the following morning for the PK determination visit.

On days 29 and 57, participants visited for new drug initiation and pharmacokinetic determination. The participants fasted for 8 hours overnight prior to attending. Blood samples were collected for:
Plasma drug concentration (24 hours post Day 28 dose)
PBMC drug concentration (24 hours post Day 28 dose)

On day 85, participants visited for pharmacokinetic determination. The participants fasted for 8 hours overnight prior to attending. Blood samples were collected for:
Plasma drug concentration (24 hours post Day 28 dose)
PBMC drug concentration (24 hours post Day 28 dose)

Pharmacokinetics

TAF

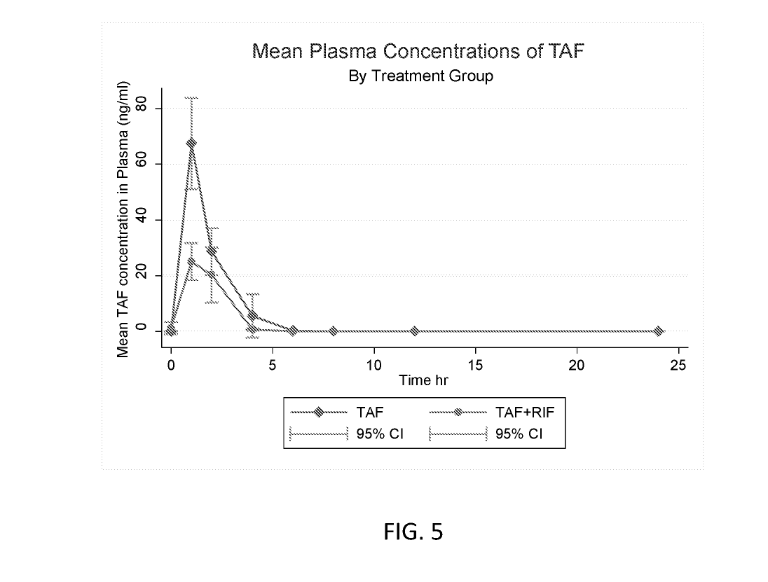

The pharmacokinetics of TAF were evaluated following administration of TAF/FTC and compared to TAF/FTC+RIF. Results are shown in Table 11 and FIG. 5.

TABLE 11
TAF plasma exposure

| | GM (95% CI) | | GMR (90% CI) |
|---|---|---|---|
| TAF PK | TAF/FTC + | | TAF/FTC + RIF |
| parameter | RIF | TAF/FTC | vs TAF/FTC |
| Cₘₐₓ (ng/mL) | 35 (27-44) | 71 (56-87) | 0.50 (0.40-0.63) |
| % CV | 53% | 48% | |
| AUC₀₋₂₄ (h*ng/mL) | 44 (33-55) | 101 (75-126) | 0.45 (0.31-0.64) |
| % CV | 55% | 57% | |

The results show that TAF plasma exposure over a 24 hour period in subjects that received TAF/FTC+RIF treatment was decreased by about 55% as compared to TAF plasma exposure in subjects that received TAF/FTC alone.

TFV

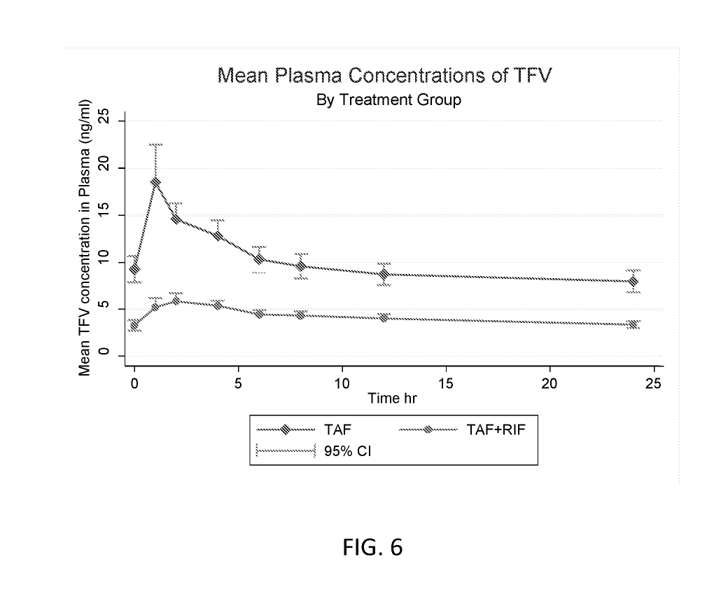

The pharmacokinetics of TFV were evaluated in the 24 hours following either TAF/FTC treatment or TAF/FTC+RIF treatment. As shown in Table 12 and FIG. 6, the total daily tenofovir (TFV) plasma exposure over a 24 hour period in subjects that received TAF/FTC+RIF treatment was decreased by about 54% as compared to TFV plasma exposure in subjects that received TAF/FTC treatment without RIF.

TABLE 12
TFV plasma exposure

| | GM (95% CI) | | GMR (90% CI) |
|---|---|---|---|
| TFV PK | TAF/FTC + | | TAF/FTC + RIF |
| parameter | RIF | TAF/FTC | vs TAF/FTC |
| Cₘₐₓ (ng/mL) | 7 (6-8) | 20 (16-24) | 0.35 (0.29-0.43) |
| % CV | 32% | 44% | |
| AUC₀₋₂₄ (h*ng/mL) | 98 (86-109) | 224 (187-261) | 0.46 (0.39-0.53) |
| % CV | 25% | 36% | |
| C_{24 h} ng/mL | 3 (3-4) | 8 (7-9) | 0.45 (0.41-0.51) |
| % CV | 28% | 34% | |

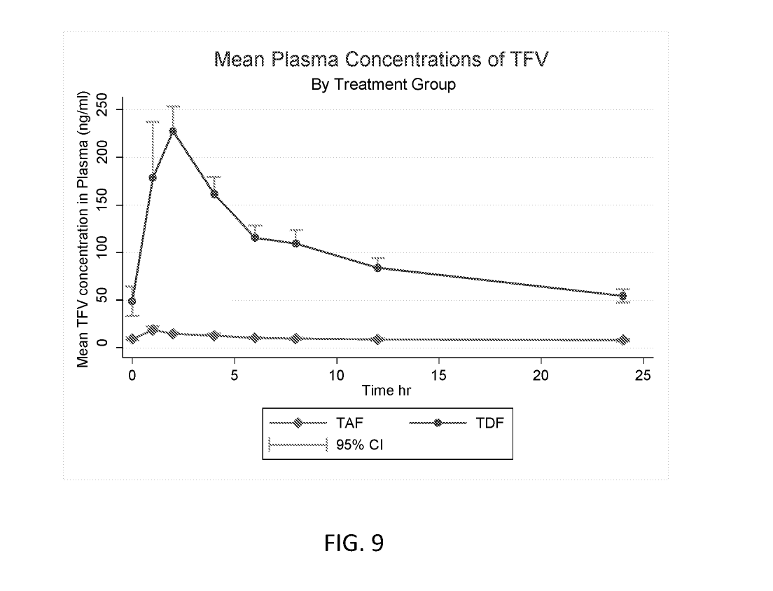

The pharmacokinetics of TFV were also evaluated in the 24 hours following either TAF/FTC treatment or TDF treatment. As shown in Table 13 and FIG. 9, the total daily TFV plasma exposure over a 24 hour period in subjects that received TAF/FTC treatment was markedly lower as compared to TFV plasma exposure in subjects that received TDF treatment alone.

TABLE 13
TFV plasma exposure

| TFV PK | GM (95% CI) | | GMR (90% CI) |
|---|---|---|---|
| parameter | TDF | TAF/FTC | TDF vs TAF/FTC |
| Cₘₐₓ (ng/mL) | 262 (222-302) | 20 (16-24) | 13.57 (11.68-15.76) |
| % CV | 33% | 44% | |
| AUC₀₋₂₄ (h*ng/mL) | 2389 (2092-2685) | 224 (187-261) | 11.10 (9.74-12.65) |
| % CV | 27% | 36% | |
| C_{24 h} ng/mL | 54 (47-62) | 8 (7-9) | 7.15 (6.44-7.93) |
| % CV | 29% | 36% | |

TFV-DP

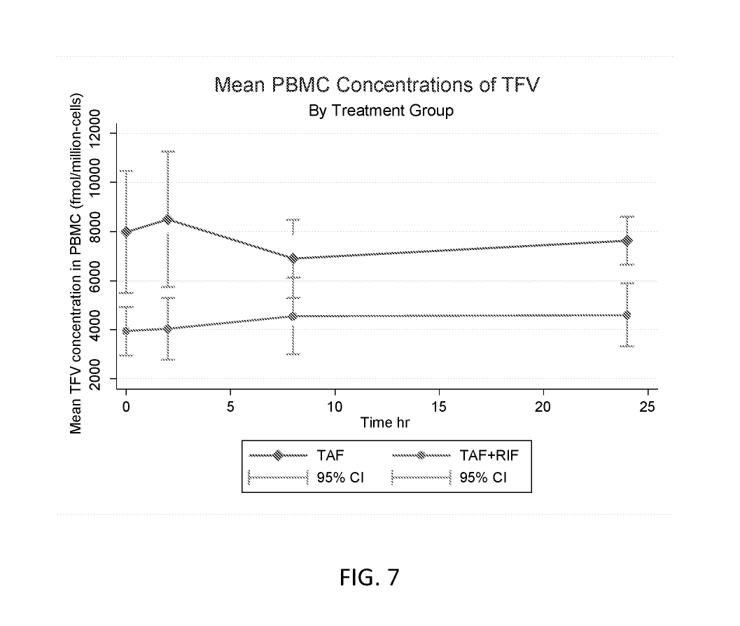

The pharmacokinetics of TFV-DP were evaluated in the 24 hours following either TAF/FTC treatment or TAF/FTC+RIF treatment. As shown in Table 14 and FIG. 7, the TFV-DP intracellular (IC) exposure over a 24 hour period in subjects that received TAF/FTC+RIF treatment was decreased by about 35% as compared to TFV-DP IC exposure in subjects that received TAF/FTC treatment without RIF.

TABLE 14
Intracellular TFV-DP exposure

| | GM (95% CI) | | GMR (90% CI) |
|---|---|---|---|
| IC TFV-DP PK | TAF/FTC + | | TAF/FTC + RIF |
| parameter | RIF | TAF/FTC | VS TAF/FTC |
| Cₘₐₓ (ng/mL) | 5966 (4240-7692) | 9582 (6643-12520) | 0.62 (0.50-0.77) |
| % CV | 64% | 67% | |
| AUC₀₋₂₄ (h*ng/mL) | 104703 (74006-135400) | 167479 (122885-212073) | 0.65 (0.53-0.79) |
| % CV | 64% | 58% | |
| C_{24 h} ng/mL | 4453 (3088-5818) | 6864 (5563-8166) | 0.57 (0.45-0.74) |
| % CV | 55% | 68% | |

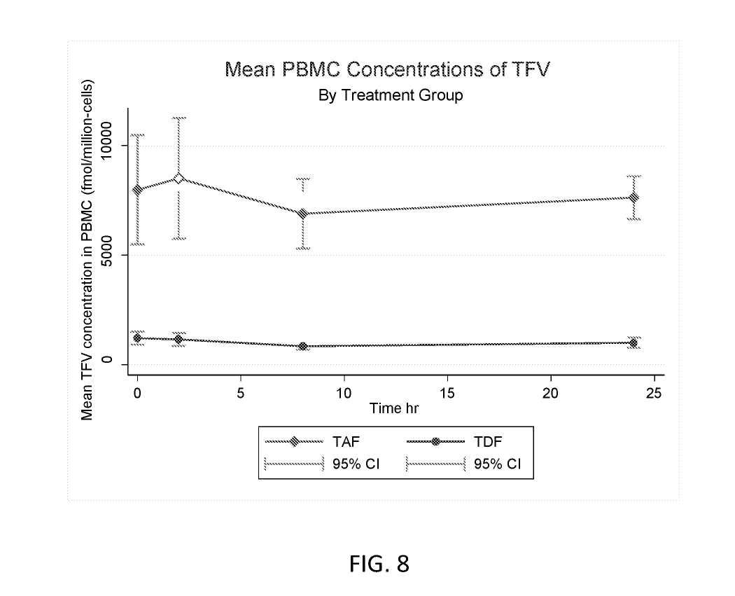

The pharmacokinetics of TFV-DP were also evaluated in the 24 hours following either TDF treatment or TAF/FTC+RIF treatment. As shown in Table 15 and FIG. 8, the TFV-DP IC exposure over a 24 hour period in subjects that received TAF/FTC+RIF treatment was increased by about 85% as compared to TFV-DP IC exposure in subjects that received TDF treatment alone.

TABLE 15
Intracellular TFV-DP exposure

| | GM (95% CI) | | GMR (90% CI) |
|---|---|---|---|
| IC TFV-DP PK | | TAF/FTC + | TDF VS TAF/ |
| parameter | TDF | RIF | FTC + RIF |
| Cₘₐₓ (ng/mL) | 1374 (1033-1715) | 5966 (4240-7692) | 0.14 (0.10-0.20) |
| % CV | 55% | 64% | |
| AUC₀₋₂₄ (h*ng/mL) | 23195 (17976-28414) | 104703 (74006-13540) | 0.15 (0.10-0.23) |
| % CV | 49% | 64% | |
| C_{24 h} ng/mL | 1004 (750-1258) | 4453 (3088-5818) | 0.14 (0.10-0.19) |
| % CV | 55% | 55% | |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating a subject co-infected with a virus and tuberculosis (TB), comprising administering a therapeutically effective amount of tenofovir alafenamide (TAF) and a therapeutically effective amount of an antimycobacterial agent to the subject, wherein the TAF is administered twice daily.

2. The method of claim 1, wherein the antimycobacterial agent is selected from the group consisting of rifampin (rifampicin; RIF), rifabutin, rifapentine, isoniazid, ethambutol, pyrazinamide, dapsone, streptomycin, p-amino-salicylate, ethionamide, cycloserine, closerin, capreomycin, viomycin, enviomycin, amikacin, kanamycin, ciprofloxacin, levofloxacin, moxifloxacin, clofazamine, ethionamide, prothionamide, clarithromycin, linezolid, thioacetazone, thioridazine, R207910, and terizidone.

3. The method of claim 1, wherein the antimycobacterial agent is RIF.

4. The method of claim 3, wherein the antimycobacterial agent is administered at a 600 mg daily dose.

5. The method of claim 4, wherein the antimycobacterial agent is administered once daily.

6. The method of claim 5, wherein the daily dose of antimycobacterial agent is administered together with the first daily dose of TAF.

7. The method of claim 6, wherein the TAF is administered at a 25 mg dose twice daily.

8. The method of claim 1, wherein the virus is selected from HIV and HBV.

9. The method of claim 8, wherein the virus is HIV.

10. The method of claim 8, wherein the virus is HBV.

11. The method of claim 7, further comprising administering one or more additional therapeutic agents selected from bictegravir, emtricitabine, elvitegravir, cobicistat, atazanavir, ritonavir, lopinavir, darunavir, rilpivirine, efavirenz, saquinavir, fosamprenavir and tipranavir.

12. The method of claim 11, wherein the one or more additional therapeutic agents are bictegravir and emtricitabine.

13. The method of claim 11, wherein the additional therapeutic agent is emtricitabine.

14. The method of claim 11, wherein at least one of the daily doses of TAF is administered together with the one or more additional therapeutic agents.

15. The method of claim 14, wherein a single tablet comprising TAF, bictegravir, and emtricitabine is administered to the subject twice daily.

16. The method of claim 15, wherein the single tablet comprises 25 mg TAF, 50 mg bictegravir, and 200 mg emtricitabine.

17. A method of treating a subject co-infected with HBV or HIV and tuberculosis (TB), comprising administering to the subject:
a single tablet twice daily comprising 25 mg TAF, 50 mg bictegravir, and 200 mg emtricitabine; and
a once daily dose of 600 mg RIF.

18. The method of claim 17, wherein the viral infection is HIV.

19. The method of claim 18 wherein the daily TAF plasma exposure is not reduced by more than about 25% compared with the daily TAF plasma exposure measured for the same treatment but wherein the TAF is administered once daily in the absence of the antimycobacterial agent.

20. The method of claim 18 wherein the daily TAF plasma exposure is not reduced by more than about 20% compared with the daily TAF plasma exposure measured for the same treatment but wherein the TAF is administered once daily in the absence of the antimycobacterial agent.

21. The method of claim 18 wherein the daily TAF plasma exposure is not reduced by more than about 15% compared with the daily TAF plasma exposure measured for the same treatment but wherein the TAF is administered once daily in the absence of the antimycobacterial agent.

22. The method of claim 21 wherein the daily tenofovir (TFV) plasma exposure is not increased by more than 20% compared with the daily tenofovir (TFV) plasma exposure measured for the same treatment but wherein the TAF is administered once daily in the absence of the antimycobacterial agent.

23. The method of claim 21 wherein the daily tenofovir (TFV) plasma exposure is not increased by more than 10% compared with the daily tenofovir (TFV) plasma exposure measured for the same treatment but wherein the TAF is administered once daily in the absence of the antimycobacterial agent.

24. The method of claim 21 wherein the daily tenofovir (TFV) plasma exposure is the same as or less than that measured for the same treatment but wherein the TAF is administered once daily in the absence of the antimycobacterial agent.

25. The method of claim 24 wherein the mean steady-state intracellular TFV-DP trough concentration is at least about 85 fmol/10⁶ cells.

26. The method of claim 24 wherein the mean steady-state intracellular TFV-DP trough concentration, is at least about 200 fmol/106 cells.

27. The method of claim 24 wherein the mean steady-state intracellular TFV-DP trough concentration, is at least about 300 fmol/10⁶ cells.

28. The method of claim 24 wherein the mean steady-state intracellular TFV-DP trough concentration, is at least about 350 fmol/10⁶ cells.

29. The method of claim 24 wherein the daily TFV-DP intracellular exposure is not reduced by more than about 30% compared with the daily TFV-DP intracellular exposure measured for the same treatment but wherein the TAF is administered once daily in the absence of the antimycobacterial agent.

30. The method of claim 24 wherein the daily TFV-DP intracellular exposure is not reduced by more than about 25% compared with the daily TFV-DP intracellular exposure measured for the same treatment but wherein the TAF is administered once daily in the absence of the antimycobacterial agent.

31. The method of claim 24 wherein the daily TFV-DP intracellular exposure is not reduced by more than about 20% compared with the daily TFV-DP intracellular exposure measured for the same treatment but wherein the TAF is administered once daily in the absence of the antimycobacterial agent.
